(19) United States
(12) Patent Application Publication
(10) Pub. No.: US 2026/0337155 A1
(43) Pub. Date: Oct. 29, 2026

(54) SOLID STATE FORMS OF MESDOPETAM AND SALTS THEREOF

(71) Applicant: ASSIA CHEMICAL INDUSTRIES LTD., Tel Aviv (IL)

(72) Inventors: Valentina TRAVANCIC, Zagreb (HR); Lorena KORDIC, Rab (HR); Helena CERIC, Zagreb (HR)

(21) Appl. No.: 19/114,056

(22) PCT Filed: Sep. 13, 2023
(86) PCT No.: PCT/IB2023/059085

§ 371 Date: Mar. 21, 2025

Related U.S. Application Data
(60) Provisional application No. 63/408,516, filed on Sep. 21, 2022.
(60) Provisional application No. 63/422,047, filed on Nov. 3, 2022.
(60) Provisional application No. 63/422,056, filed on Nov. 3, 2022.

(51) Int. Cl. C07C 309/42

(52) U.S. Cl. CPC C07C 309/42; C07B 2200/13

(57) ABSTRACT

The present disclosure encompasses Mesdopetam salts, solid state forms of Mesdopetam and salts thereof, in embodiments processes for preparation thereof and pharmaceutical compositions thereof.

### FIELD OF THE DISCLOSURE

The present disclosure encompasses Mesdopetam salts, solid state forms of Mesdopetam and salts thereof, in embodiments processes for preparation thereof and pharmaceutical compositions thereof.

### BACKGROUND OF THE DISCLOSURE

Mesdopetam, N-[2-(3-fluoro-5-methylsulfonylphenoxy)ethyl]propan-1-amine, has the following chemical structure:

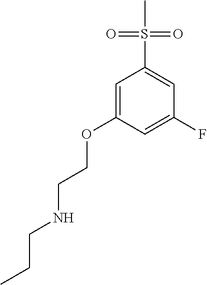 [SMILES: CCCNCCOc1cc(F)cc(S(C)(=O)=O)c1]

Mesdopetam is developed for the treatment of levodopa-induced dyskinesias commonly occurring in Parkinson's disease. Mesdopetam is also being investigated for the treatment or Parkinson's disease psychosis.

The compound is described in International Publication No. WO 2012/143337

Mesdopetam salts are described in International Publication No. WO 2020/239568.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new salts and solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Mesdopetam.

### SUMMARY OF THE DISCLOSURE

The present disclosure provides Mesdopetam salts, or crystalline polymorphs of Mesdopetam and salts thereof, processes for preparation thereof and pharmaceutical composition thereof. Any one of the crystalline forms can be used to prepare other solid state forms of Mesdopetam and Mesdopetam salts and solid state forms thereof.

The present disclosure also provides uses of said Mesdopetam salts, or solid state forms of Mesdopetam or salts thereof in the preparation of other solid state forms of Mesdopetam or salts thereof.

The present disclosure provides said Mesdopetam salts, or crystalline forms of Mesdopetam and salts thereof for use in medicine, including for the treatment of patients with Parkinson's Disease Dyskinesia, levodopa-induced dyskinesias, or Parkinson's disease Psychosis.

The present disclosure also encompasses the use of said Mesdopetam salts, or crystalline polymorphs of Mesdopetam and salts thereof for the preparation of pharmaceutical compositions and/or formulations.

In another aspect, the present disclosure provides pharmaceutical compositions comprising said Mesdopetam salts, or crystalline polymorphs of Mesdopetam or salts thereof according to the present disclosure.

The present disclosure includes processes for preparing the above mentioned pharmaceutical compositions. The processes include combining any one or a combination of the Mesdopetam salts, or crystalline polymorphs of Mesdopetam or salts thereof with at least one pharmaceutically acceptable excipient.

The crystalline polymorphs of Mesdopetam or salts thereof as defined herein and the pharmaceutical compositions or formulations of the Mesdopetam salts, or crystalline polymorphs of Mesdopetam or salts thereof may be used as medicaments, such as for the treatment of patients with Parkinson's Disease Dyskinesia, levodopa-induced dyskinesias, or Parkinson's disease Psychosis.

The present disclosure also provides methods for the treatment of patients with levodopa-induced dyskinesias by administering a therapeutically effective amount of said Mesdopetam salts, or crystalline polymorphs of Mesdopetam or salts thereof of the present disclosure, or at least one of the above pharmaceutical compositions, to a subject suffering from Parkinson's Disease Dyskinesia, levodopa-induced dyskinesia, or Parkinson's disease Psychosis or otherwise in need of the treatment.

The present disclosure also provides uses of said Mesdopetam salts, or crystalline polymorphs of Mesdopetam or salts thereof of the present disclosure, or at least one of the above pharmaceutical compositions, for the manufacture of medicaments for treating e.g., patients with Parkinson's Disease Dyskinesia, levodopa-induced dyskinesia, or Parkinson's disease Psychosis.

### BRIEF DESCRIPTION OF THE DRAWINGS

FIG. 1 shows a characteristic X-ray powder diffraction pattern (XRPD) of Mesdopetam Form B1.

FIG. 2 shows the X-ray powder diffraction pattern (XRPD) of Mesdopetam Form B3 prepared according to Example 2.

FIG. 3 shows the X-ray powder diffraction pattern (XRPD) of Mesdopetam Form B4 prepared according to Example 3.

FIG. 4 shows the X-ray powder diffraction pattern (XRPD) of Mesdopetam mesylate Form MS1 prepared according to Example 4.

FIG. 5 shows the X-ray powder diffraction pattern (XRPD) of Mesdopetam mesylate Form MS2 prepared according to Example 5, procedure A.

FIG. 6 shows the X-ray powder diffraction pattern (XRPD) of Mesdopetam besylate Form BS1 prepared according to Example 6.

FIG. 7 shows the X-ray powder diffraction pattern (XRPD) of Mesdopetam (L)-malate Form M1 prepared according to Example 7.

FIG. 8 shows the X-ray powder diffraction pattern (XRPD) of Mesdopetam hemi-(L)-malate Form HMI prepared according to Example 8.

### DETAILED DESCRIPTION OF THE DISCLOSURE

The present disclosure encompasses Mesdopetam salts, or crystalline forms of Mesdopetam and salts thereof, processes for preparation thereof, and pharmaceutical compositions thereof.

Solid state properties of Mesdopetam or salts thereof and crystalline polymorphs thereof can be influenced by controlling the conditions under which Mesdopetam and crystalline polymorphs thereof are obtained in solid form.

The solid state forms of Mesdopetam (e.g. Mesdopetam, Mesdopetam salts) as described in any aspect or embodiment of the present disclosure may be polymorphically pure, or substantially free of any other solid state (or polymorphic) forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, polymorphically pure Mesdopetam form B1 means that the solid state form is substantially free of other solid state forms of Mesdopetam. Thus, a crystalline polymorph of Mesdopetam or salts thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Mesdopetam or salts thereof. In some embodiments of the disclosure, the described crystalline polymorph of Mesdopetam, salt or cocrystal may contain from about 1% to about 20% (w/w), from about 5% to about 20% (w/w), or from about 5% to about 10% (w/w) of one or more other crystalline polymorph of the same Mesdopetam.

Depending on which other crystalline polymorphs a comparison is made, the crystalline polymorphs of Mesdopetam or salts thereof of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Mesdopetam referred to herein as being characterized by graphical data “as depicted in” or “as substantially depicted in” a Figure will thus be understood to include any crystal forms of Mesdopetam characterized with the graphical data having such small variations, as are well known to the skilled person, in comparison with the Figure.

As used herein, and unless stated otherwise, the term “anhydrous” in relation to crystalline forms of Mesdopetam relates to a crystalline form of Mesdopetam which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Mesdopetam of the present disclosure corresponds to a crystalline polymorph of Mesdopetam that is physically separated from the reaction mixture in which it is formed.

As used herein, unless stated otherwise, the XRPD measurements are taken using copper K α radiation wavelength 1.54184 Å. XRPD peaks reported herein are optionally measured using CuK α radiation, λ=1.54184 Å, typically at a temperature of 25±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The Mesdopetam salts, or solid state form of Mesdopetam (e.g. Mesdopetam, Mesdopetam salts) as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w) or less, about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Mesdopetam or salts thereof described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2% (w/w), greater than about 99.4% (w/w), greater than about 99.6% (w/w), greater than about 99.8% (w/w), greater than about 99.9% (w/w), or about 100% of the subject Mesdopetam or salts thereof. Alternatively, pure or purified Mesdopetam or salts thereof, described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2% area percent, greater than about 99.4% area percent, greater than about 99.6% area percent, greater than about 99.8% area percent, greater than about 99.9% area percent, or about 100% of the subject Mesdopetam or salts thereof.

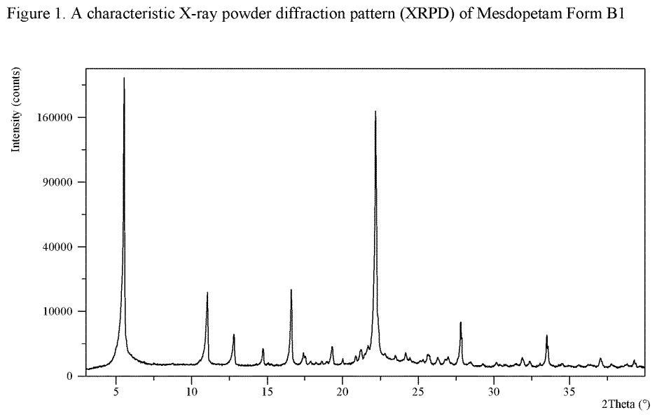

The present disclosure includes a crystalline polymorph Mesdopetam designated Form B1. The crystalline Form B1 of Mesdopetam may be characterized by data selected from one or more of the following: an X-ray powder diffraction pattern substantially as depicted in FIG. 1; an X-ray powder diffraction pattern having peaks at 5.5, 11.1, 16.6 and 22.2 degrees 2-theta±0.2 degrees 2-theta; and combinations of these data.

Crystalline Form B1 of Mesdopetam may be further characterized by an X-ray powder diffraction pattern having peaks at 5.5, 11.1, 16.6 and 22.2 degrees 2-theta±0.2 degrees 2-theta, and also having any one, two, three, four or five additional peaks selected from 12.8, 14.7, 19.3 and 27.8 degrees 2-theta±0.2 degrees 2-theta.

Crystalline Form B1 of Mesdopetam may be alternatively characterized by an X-ray powder diffraction pattern having peaks at 5.5, 11.1, 12.8, 14.7, 16.6, 19.3, 22.2 and 27.8 degrees 2-theta±0.2 degrees 2-theta.

In any aspect or embodiment of the present disclosure, crystalline Form B1 of Mesdopetam may be isolated. Particularly, crystalline Form B1 of Mesdopetam according to any aspect or embodiment of the disclosure may be isolated.

In any aspect or embodiment crystalline form B1 may be chemically pure.

In any aspect or embodiment crystalline Form B1 of Mesdopetam may be polymorphically pure.

In any aspect or embodiment of the present disclosure, crystalline Form B1 of Mesdopetam may be anhydrous.

Crystalline Form B1 of Mesdopetam may be characterized by each of the above characteristics alone/or by all possible combinations, e.g., an XRPD pattern having peaks at 5.5, 11.1, 16.6 and 22.2 degrees 2-theta±0.2 degrees 2-theta; an XRPD pattern as depicted in FIG. 1, and combinations thereof.

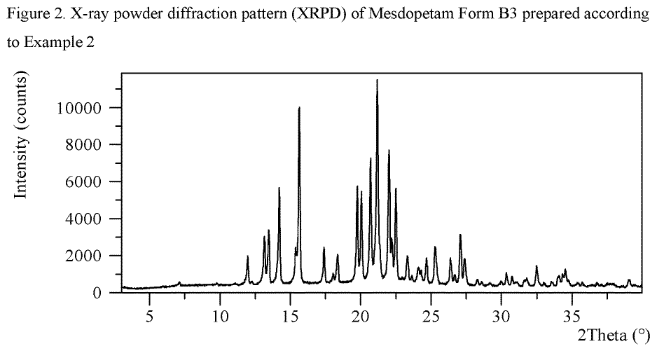

The present disclosure includes a crystalline polymorph Mesdopetam designated Form B3. The crystalline Form B3 of Mesdopetam may be characterized by data selected from one or more of the following: an X-ray powder diffraction pattern substantially as depicted in FIG. 2; an X-ray powder diffraction pattern having peaks at 12.0, 13.5, 14.2 and 18.4 degrees 2-theta±0.2 degrees 2-theta; and combinations of these data.

Crystalline Form B3 of Mesdopetam may be further characterized by an X-ray powder diffraction pattern having peaks at 12.0, 13.5, 14.2 and 18.4 degrees 2-theta±0.2 degrees 2-theta, and also having any one, two, three or four additional peaks selected from 17.4, 23.3, 25.3 and 27.1 degrees 2-theta±0.2 degrees 2-theta.

Crystalline Form B3 of Mesdopetam may be alternatively characterized by an X-ray powder diffraction pattern having peaks at 12.0, 13.5, 14.2, 17.4, 18.4, 23.3, 25.3 and 27.1 degrees 2-theta±0.2 degrees 2-theta.

In any aspect or embodiment of the present disclosure, crystalline Form B3 of Mesdopetam may be isolated. Particularly, crystalline Form B3 of Mesdopetam according to any aspect or embodiment of the disclosure may be isolated.

In any aspect or embodiment crystalline form B3 may be chemically pure.

In any aspect or embodiment crystalline Form B3 of Mesdopetam may be polymorphically pure.

Crystalline Form B3 of Mesdopetam may be characterized by each of the above characteristics alone/or by all possible combinations, e.g., an XRPD pattern having peaks at 12.0, 13.5, 14.2 and 18.4 degrees 2-theta±0.2 degrees 2-theta; an XRPD pattern as depicted in FIG. 2, and combinations thereof.

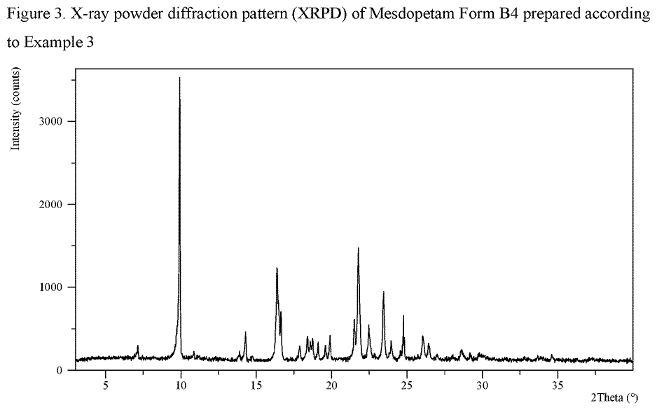

The present disclosure includes a crystalline polymorph Mesdopetam designated Form B4. The crystalline Form B4 of Mesdopetam may be characterized by data selected from one or more of the following: an X-ray powder diffraction pattern substantially as depicted in FIG. 3; an X-ray powder diffraction pattern having peaks at 9.9, 16.4, 21.8, 23.4 and 26.0 degrees 2-theta±0.2 degrees 2-theta; and combinations of these data.

Crystalline Form B4 of Mesdopetam may be further characterized by an X-ray powder diffraction pattern having peaks at 9.9, 16.4, 21.8, 23.4 and 26.0 degrees 2-theta±0.2 degrees 2-theta, and also having any one, two, three or four additional peaks selected from 7.1, 14.3, 17.9, 19.9 and 22.5 degrees 2-theta±0.2 degrees 2-theta.

Crystalline Form B4 of Mesdopetam may be alternatively characterized by an X-ray powder diffraction pattern having peaks at 7.1, 9.9, 14.3, 16.4, 17.9, 19.9, 21.8, 22.5, 23.4 and 26.0 degrees 2-theta±0.2 degrees 2-theta.

In any aspect or embodiment of the present disclosure, crystalline Form B4 of Mesdopetam may be isolated. Particularly, crystalline Form B4 of Mesdopetam according to any aspect or embodiment of the disclosure may be isolated.

In any aspect or embodiment crystalline form B4 may be chemically pure.

In any aspect or embodiment crystalline Form B4 of Mesdopetam may be polymorphically pure.

Crystalline Form B4 of Mesdopetam may be characterized by each of the above characteristics alone/or by all possible combinations, e.g., an XRPD pattern having peaks at 9.9, 16.4, 21.8, 23.4 and 26.0 degrees 2-theta±0.2 degrees 2-theta; an XRPD pattern as depicted in FIG. 3, and combinations thereof.

The present disclosure encompasses Mesdopetam mesylate. The Mesdopetam mesylate is preferably in a solid form, and more preferably the Mesdopetam mesylate is crystalline. In any aspect or embodiment of the present disclosure, Mesdopetam mesylate may be isolated. Particularly, Mesdopetam mesylate according to any aspect or embodiment may be chemically pure. Crystalline Mesdopetam mesylate according to any aspect or embodiment may be polymorphically pure (i.e. containing a single crystalline form of Mesdopetam mesylate), preferably containing about 20% (w/w) or less, about 10% (w/w) or less, about 5% (w/w) or less, about 2% (w/w) or less, about 1% (w/w) or less, or about 0% of any other crystalline form of Mesdopetam mesylate, as measured, for example, by XRPD. In any aspect or embodiment of the present disclosure, Mesdopetam mesylate may be anhydrous.

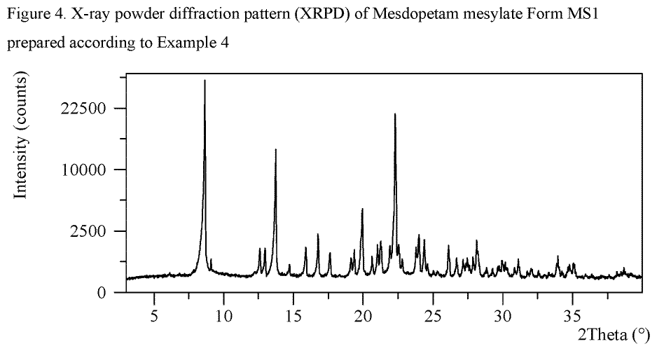

The present disclosure includes a crystalline polymorph Mesdopetam mesylate designated Form MS1. The crystalline Form MS1 of Mesdopetam mesylate may be characterized by data selected from one or more of the following: an X-ray powder diffraction pattern substantially as depicted in FIG. 4; an X-ray powder diffraction pattern having peaks at 8.7, 13.7, 16.8, 19.9 and 22.3 degrees 2-theta±0.2 degrees 2-theta; and combinations of these data.

Crystalline Form MS1 of Mesdopetam mesylate may be further characterized by an X-ray powder diffraction pattern having peaks at 8.7, 13.7, 16.8, 19.9 and 22.3 degrees 2-theta±0.2 degrees 2-theta, and also having any one, two, three, four or five additional peaks selected from 12.6, 13.0, 15.9, 17.6 and 26.1 degrees 2-theta±0.2 degrees 2-theta.

Crystalline Form MS1 of Mesdopetam mesylate may be alternatively characterized by an X-ray powder diffraction pattern having peaks at 8.7, 12.6, 13.0, 13.7, 15.9, 16.8, 17.6, 19.9, 22.3 and 26.1 degrees 2-theta±0.2 degrees 2-theta.

In any aspect or embodiment of the present disclosure, crystalline Form MS1 of Mesdopetam mesylate may be isolated. Particularly, crystalline Form MS1 of Mesdopetam mesylate according to any aspect or embodiment of the disclosure may be isolated.

In any aspect or embodiment crystalline form MS1 of Mesdopetam mesylate may be chemically pure.

In any aspect or embodiment crystalline Form MS1 of Mesdopetam mesylate may be polymorphically pure.

In any aspect or embodiment of the present disclosure, crystalline Form MS1 of Mesdopetam mesylate may be anhydrous.

Crystalline Form MS1 of Mesdopetam mesylate may be characterized by each of the above characteristics alone/or by all possible combinations, e.g., an XRPD pattern having peaks at 8.7, 13.7, 16.8, 19.9 and 22.3 degrees 2-theta±0.2 degrees 2-theta; an XRPD pattern as depicted in FIG. 4, and combinations thereof.

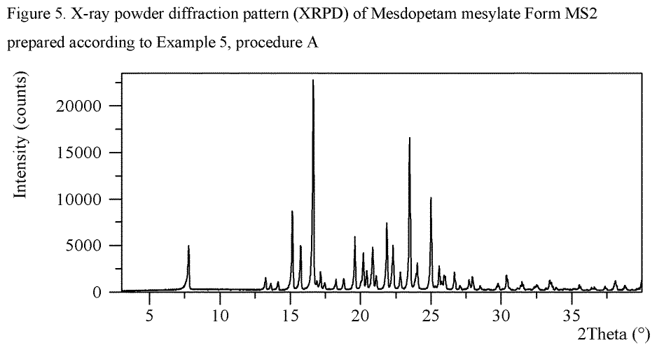

The present disclosure includes a crystalline polymorph Mesdopetam mesylate designated Form MS2. The crystalline Form MS2 of Mesdopetam mesylate may be characterized by data selected from one or more of the following: an X-ray powder diffraction pattern substantially as depicted in FIG. 5; an X-ray powder diffraction pattern having peaks at 7.8, 15.2, 19.6, 23.5 and 25.0 degrees 2-theta±0.2 degrees 2-theta; and combinations of these data.

Crystalline Form MS2 of Mesdopetam mesylate may be further characterized by an X-ray powder diffraction pattern having peaks at 7.8, 15.2, 19.6, 23.5 and 25.0 degrees 2-theta±0.2 degrees 2-theta, and also having any one, two, three, four or five additional peaks selected from 15.7, 17.2, 18.2, 20.2 and 20.9 degrees 2-theta±0.2 degrees 2-theta.

Crystalline Form MS2 of Mesdopetam mesylate may be alternatively characterized by an X-ray powder diffraction pattern having peaks at 7.8, 15.2, 15.7, 17.2, 18.2, 19.6, 20.2, 23.5 and 25.0 degrees 2-theta±0.2 degrees 2-theta.

Crystalline Form MS2 of Mesdopetam mesylate may be alternatively characterized by an X-ray powder diffraction pattern having peaks at 7.8, 15.2, 15.7, 17.2, 18.2, 19.6, 20.2, 20.9, 23.5 and 25.0 degrees 2-theta±0.2 degrees 2-theta.

In any aspect or embodiment of the present disclosure, crystalline Form MS2 of Mesdopetam mesylate may be isolated. Particularly, crystalline Form MS2 of Mesdopetam mesylate according to any aspect or embodiment of the disclosure may be isolated.

In any aspect or embodiment crystalline form MS2 of Mesdopetam mesylate may be chemically pure.

In any aspect or embodiment crystalline Form MS2 of Mesdopetam mesylate may be polymorphically pure.

In any aspect or embodiment of the present disclosure, crystalline Form MS2 of Mesdopetam mesylate may be anhydrous.

Crystalline Form MS2 of Mesdopetam mesylate may be characterized by each of the above characteristics alone/or by all possible combinations, e.g., an XRPD pattern having peaks at 7.8, 15.2, 19.6, 23.5 and 25.0 degrees 2-theta±0.2 degrees 2-theta; an XRPD pattern as depicted in FIG. 5, and combinations thereof.

Mesdopetam Mesylate may be prepared by reacting Mesdopetam with methane sulfonic acid in any suitable solvent. The solvent is preferably selected at least enable dissolution of Mesdopetam and methanesulfonic acid, and optionally enables precipitation of the resulting Mesdopetam Mesylate (e.g. through cooling and/or evaporation). If the solvent does not readily enable precipitation of Mesdopetam Mesylate, an antisolvent for Mesdopetam Mesylate, for example a non-polar solvent such as an alkane, or a solvent selected from n-heptane, dichloromethane, acetone or tetrahydrofuran, may be added to facilitate precipitation.

Crystalline form MS2 of Mesdopetam Mesylate may be prepared by crystallisation of Mesdopetam Mesylate from a solvent comprising isopropanol, acetonitrile, ethanol, methanol, or mixture thereof. In any embodiment, the process may comprise:
(a) providing a solution of Mesdopetam Mesylate in a solvent, preferably wherein the solvent comprises acetonitrile, ethanol, methanol, or mixtures thereof;
(b) crystallising Mesdopetam Mesylate Form MS2; and
(c) optionally isolating crystalline form MS2 of Mesdopetam Mesylate from the mixture.

In any embodiment of this process, the solution in step (a) may be prepared by combining Mesdopetam mesylate with the solvent. In any embodiment of the process, the solution in step (a) may be at elevated temperature, preferably, at a temperature of about 35° C. to about 75° C., about 40° C. to about 70° C., or about 45° C. to about 65° C. In any embodiment, the solution in step (a) may be prepared by combining Mesdopetam mesylate with the heated solvent, or by combining Mesdopetam mesylate with the solvent at ambient temperature, and heating the mixture to provide a solution. In any embodiment of the process, step (b) may comprise: cooling the mixture (preferably to about 5° C. to about 30° C., or about 10° C. to about 25° C. or about 20° C. to about 25° C.). Preferably, in any embodiment of this process, the solution in step (a) is heated to a temperature of about 40° C. to about 70° C. (preferably about 45° C. to about 65° C.), and step (b) comprises cooling the mixture in step (a) to a temperature of: about 5° C. to about 35° C., about 10° C. to about 30° C., or room temperature. In any embodiment, the cooling may be active or passive, preferably passive (i.e. the mixture is allowed to cool under ambient conditions). In any embodiment of this process, the cooling is preferably conducted without stirring. In any embodiment of this process, step (c) may comprise isolating the solid by any suitable method such as by decantation, centrifuge or by filtration, preferably by filtration. The Mesdopetam mesylate Form MS2 may optionally be dried, for example under reduced pressure, and optionally elevated temperature, preferably at reduced pressure and at a temperature of: about 40° C. to about 90° C., about 55° C. to about 85° C., or about 60° C. to about 80° C.

In any embodiment, the process may comprise:
(a) providing a solution of Mesdopetam Mesylate in a solvent comprising, acetonitrile, ethanol, methanol, or mixtures thereof at elevated temperature, preferably at a temperature of about 40° C. to about 70° C. or preferably about 45° C. to about 65° C.;
(b) cooling the mixture to about 30° C., or room temperature (preferably by allowing to cool); and
(c) optionally isolating crystalline form MS2 of Mesdopetam Mesylate from the mixture. Preferably, according to any aspect or embodiment of the processes, when the solvent is acetonitrile, step a) may be performed at a temperature of about 40° C. to about 80° C., about 50° C. to about 70° C. or about 55° C. to about 65° C.; when the solvent is ethanol, step a) may be performed at a temperature of about 40° C. to about 70° C., about 45° C. to about 60° C. or about 50° C. to about 60° C.; and when the solvent is methanol, step a) may be performed at a temperature of times be dried, for example under reduced pressure, and optionally elevated temperature, preferably at reduced pressure and at a temperature of: about 40° C. to about 90° C., about 55° C. to about 85° C., or about 60° C. to about 80° C.

Alternatively, Mesdopetam mesylate form MS2 may be prepared by a process comprising stirring a suspension of Mesdopetam mesylate in isopropanol. Preferably, the suspension of Mesdopetam mesylate in isopropanol is prepared by combining Mesdopetam (preferably form B1), methanesulfonic acid, and isopropanol. More particularly, the process comprises:
(i) providing a mixture of Mesdopetam, preferably form B1, and methanesulfonic acid in isopropanol;
(ii) optionally stirring the mixture; and
(iii) optionally isolating crystalline form MS2 of Mesdopetam Mesylate from the mixture.

In any aspect or embodiment of this process, in step (i), the isopropanol may be present in an amount of: about 5 to about 20 ml per gram of Mesdopetam, about 7 to about 15 ml per gram of Mesdopetam, or about 7 to about 12 ml per gram of Mesdopetam. In any embodiment of this process, the molar ratio of methanesulfonic acid to Mesdopetam may be: about 1 to about 1.5 molar equivalents, about 1 to about 1.3 molar equivalents, about 1.0 to about 1.2 molar equivalents, about 1.1 molar equivalents, or about 1.0 molar equivalents. In any embodiment of this process, the mixture in step (i) is preferably at room temperature. In any embodiment of this process, step (ii) comprises stirring the mixture for a sufficient time to prepare Mesdopetam mesylate form MS2, preferably about 8 hours to about 4 days, about 12 hours to about 72 hours, about 20 hours to about 60 hours, or about 24 hours to about 48 hours. In any embodiment of this process, step (iii) comprises isolating the Mesdopetam mesylate Form MS2 by any suitable method such as by decantation, centrifuge or filtration, preferably filtration. The Mesdopetam mesylate Form MS2 may optionally be dried, for example under reduced pressure, and optionally elevated temperature, preferably at reduced pressure and at a temperature of: about 40° C. to about 90° C., about 55° C. to about 85° C., or about 60° C. to about 80° C.; optionally, the drying can be conducted for a sufficient time to obtain a dried solid, preferably about 1 hour to about 10 hours, about 2 hours to about 8 hours, about 3 hours to about 6 hours, or about 4 hours to about 6 hours.

Crystalline form MS2 of Mesdopetam Mesylate may alternatively be prepared by crystallisation of Mesdopetam Mesylate from water. In any embodiment, the process may comprise:
(a) providing a solution comprising Mesdopetam Mesylate in water;
(b) crystallising Mesdopetam Mesylate Form MS2 from the solution; and
(c) optionally isolating crystalline form MS2 of Mesdopetam Mesylate.

In any embodiment of this process, the solution in step (a) may be prepared by combining Mesdopetam (preferably form B1), methanesulfonic acid, and water. In any embodiment of this process, in step (a), the water may be present in an amount of: about 5 to about 20 ml per gram of Mesdopetam, about 10 to about 18 ml per gram of Mesdopetam, about 12 to about 16 ml per gram of Mesdopetam or about 13 to about 14 ml per gram of Mesdopetam. In any embodiment of this process, the molar ratio of methanesulfonic acid to Mesdopetam may be: about 1 to about 1.5 molar equivalents, about 1 to about 1.3 molar equivalents, about 1.0 to about 1.2 molar equivalents, about 1.1 molar equivalents, or about 1.0 molar equivalents. In any embodiment of this process, the solution in step (a) is preferably at room temperature. In any embodiment of this process, step (b) comprises stirring the mixture, preferably about 1 to about 60 minutes, about 2 to about 30 minutes, about 2 to about 10 minutes, or about 5 minutes. In any embodiment of this process, step (c) comprises partial or complete evaporation of the water, preferably by exposure to room temperature. Preferably, in any embodiment of this process, step (c) comprises allowing the water to evaporate over a period of about 1 to about 10 days, about 2 to about 8 days, about 3 to about 6 days, or about 4 days. The Mesdopetam mesylate may optionally be isolated by any suitable process if the water is partially evaporated, preferably by decantation, centrifuge or filtration, preferably filtration. The Mesdopetam mesylate Form MS2 may optionally be dried, for example under reduced pressure, and optionally elevated temperature, preferably at reduced pressure and at a temperature of: about 40° C. to about 90° C., about 55° C. to about 85° C., or about 60° C. to about 80° C.

The present disclosure encompasses Mesdopetam besylate. The Mesdopetam besylate is preferably in a solid form, and more preferably the Mesdopetam besylate is crystalline. In any aspect or embodiment of the present disclosure, Mesdopetam besylate may be isolated. Particularly, Mesdopetam besylate according to any aspect or embodiment may be chemically pure. Crystalline Mesdopetam besylate according to any aspect or embodiment may be polymorphically pure (i.e. containing a single crystalline form of Mesdopetam besylate), preferably containing about 20% (w/w) or less, about 10% (w/w) or less, about 5% (w/w) or less, about 2% (w/w) or less, about 1% (w/w) or less, or about 0% of any other crystalline form of Mesdopetam besylate, as measured, for example, by XRPD. In any aspect or embodiment of the present disclosure, Mesdopetam besylate may be anhydrous.

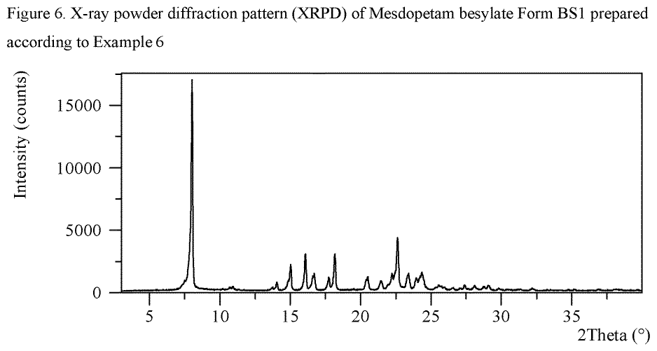

The present disclosure includes a crystalline polymorph Mesdopetam besylate designated Form BS1. The crystalline Form BS1 of Mesdopetam besylate may be characterized by data selected from one or more of the following: an X-ray powder diffraction pattern substantially as depicted in FIG. 6; an X-ray powder diffraction pattern having peaks at 8.0, 15.0, 16.1, 18.2 and 22.6 degrees 2-theta±0.2 degrees 2-theta; and combinations of these data.

Crystalline Form BS1 of Mesdopetam besylate may be further characterized by an X-ray powder diffraction pattern having peaks at 8.0, 15.0, 16.1, 18.2 and 22.6 degrees 2-theta±0.2 degrees 2-theta, and also having any one, two, three or four additional peaks selected from 16.7, 17.7, 20.5 and 23.4 degrees 2-theta±0.2 degrees 2-theta.

Crystalline Form BS1 of Mesdopetam besylate may be alternatively characterized by an X-ray powder diffraction pattern having peaks at 8.0, 15.0, 16.1, 16.7, 17.7, 18.2, 20.5, 22.6 and 23.4 degrees 2-theta±0.2 degrees 2-theta.

In any aspect or embodiment of the present disclosure, crystalline Form BS1 of Mesdopetam besylate may be isolated. Particularly, crystalline Form BS1 of Mesdopetam besylate according to any aspect or embodiment of the disclosure may be isolated.

In any aspect or embodiment crystalline form BS1 of Mesdopetam besylate may be chemically pure.

In any aspect or embodiment crystalline Form BS1 of Mesdopetam besylate may be polymorphically pure.

In any aspect or embodiment of the present disclosure, crystalline Form BS1 of Mesdopetam besylate may be anhydrous.

Crystalline Form BS1 of Mesdopetam besylate may be characterized by each of the above characteristics alone/or by all possible combinations, e.g., an XRPD pattern having peaks at 8.0, 15.0, 16.1, 18.2 and 22.6 degrees 2-theta±0.2 degrees 2-theta; an XRPD pattern as depicted in FIG. 6, and combinations thereof.

Mesdopetam Besylate may be prepared by reacting Mesdopetam with benzenesulfonic acid in any suitable solvent. The solvent is preferably selected at least enable dissolution of Mesdopetam and benzenesulfonic acid, and optionally enables precipitation of the resulting Mesdopetam Besylate (e.g. by cooling or evaporation). If the solvent does not readily enable precipitation of Mesdopetam Besylate, an antisolvent for Mesdopetam Besylate, for example a non-polar solvent such as an alkane, or a solvent selected from n-heptane, dichloromethane, acetone or tetrahydrofuran, may be added to facilitate precipitation.

Crystalline form BS1 of Mesdopetam Besylate may be prepared by crystallisation from a mixture comprising Mesdopetam Besylate and a solvent comprising isopropanol, water, acetonitrile, ethanol, methanol, or mixture thereof. In embodiments, the process comprises:
(a) providing a solution of Mesdopetam Besylate in a solvent comprising acetonitrile, ethanol, methanol, isopropanol, water or mixtures thereof;
(b) cooling the mixture; and
(c) optionally isolating crystalline form BS1 of Mesdopetam Besylate from the mixture.

In any embodiment of this process, the solution in step (a) may be prepared by combining Mesdopetam besylate with the solvent. In any embodiment, the solvent may be isopropanol, water, acetonitrile, ethanol (optionally 96% ethanol/water or absolute ethanol), or methanol, or a mixture of acetonitrile and water. According to any embodiment, in step (a), the solvent may be present in an amount of: about 2 ml to about 40 ml per gram of Mesdopetam Besylate, about 4 ml to about 35 ml per gram of Mesdopetam Besylate, or about 5 to about 30 ml per gram of Mesdopetam Besylate. In any embodiment of the process, the solution in step (a) may be at elevated temperature, preferably, at a temperature of about 15° C. to about 70° C., about 18° C. to about 65° C., about 20° C. to about 60° C., or about 22° C. to about 58° C. In any embodiment, the solution in step (a) may be prepared by combining Mesdopetam besylate with the heated solvent, or by combining Mesdopetam besylate with the solvent at ambient temperature, and optionally heating the mixture to provide a solution. In any embodiment of the process, step (b) may comprise: cooling the mixture, preferably to about 5° C. to about 30° C., or about 10° C. to about 25° C. or about 20° C. to about 25° C. Preferably, in any embodiment of this process, the solution in step (a) is heated to a temperature of about 18° C. to about 60° C. (preferably about 22° C. to about 60° C.), and step (b) comprises cooling the mixture in step (a) to a temperature of: about 5° C. to about 35° C., about 10° C. to about 30° C., or room temperature. In any embodiment, the cooling may be active or passive, preferably passive (i.e. the mixture is allowed to cool under ambient conditions). In any embodiment of this process, the cooling is preferably conducted without stirring. In any embodiment of this process, step (c) may comprise isolating the solid by any suitable method such as by decantation, centrifuge or by filtration, preferably by filtration. The Mesdopetam Besylate Form BS1 may optionally be dried, for example under reduced pressure, and optionally elevated temperature, preferably at reduced pressure and at a temperature of: about 40° C. to about 90° C., about 55° C. to about 85° C., or about 60° C. to about 80° C. According to any embodiment of the process, when the solvent is acetonitrile, the solution in step a) may be at a temperature of about 30° C. to about 70° C., about 40° C. to about 60° C., about 45° C. to about 55° C., or about 45° C. to about 50° C.; and/or the acetonitrile is used in an amount of about 10 ml to about 30 ml, about 15 ml to 25 ml, or about 20 ml per gram of Mesdopetam Besylate. According to any embodiment of the process, when the solvent is ethanol, step a) may be performed at a temperature of about 40° C. to about 70° C., about 45° C. to about 60° C., about 50° C. to about 60° C. or about 55° C. to about 60°; and/or the ethanol is used in an amount of about 15 ml to 25 ml, or about 20 ml per gram of Mesdopetam Besylate. According to any embodiment of the process, when the solvent is methanol, step a) may be performed at a temperature of about 18° C. to about 35° C., about 18° C. to about 30° C., about 20° C. to about 25° C. or at room temperature; and/or the methanol is used in an amount of about 5 ml to 20 ml, about 8 to about 15 ml, or about 10 ml per gram of Mesdopetam Besylate. According to any embodiment of the process, when the solvent is isopropanol, step a) may be performed at a temperature of about 30° C. to about 70° C., about 40° C. to about 60° C., about 45° C. to about 55° C., or about 48° C. to about 54° C.; and/or the isopropanol is used in an amount of about 15 ml to 50 ml, about 20 ml to about 40 ml, about 25 ml to about 35 ml, or about 30 ml per gram of Mesdopetam Besylate. According to any embodiment of the process, when the solvent a mixture of acetonitrile and water, step a) may be performed at a temperature of about 18° C. to about 35° C., about 18° C. to about 30° C., about 20° C. to about 25° C. or at room temperature; and/or the combined amount of acetonitrile/water used is: about 2 ml to 15 ml, about 2 ml to about 10 ml, about 2 ml to about 8 ml, 4 ml to about 7 ml, or about 5 ml per gram of Mesdopetam Besylate. Preferably, in any embodiment of the process wherein a mixture of acetonitrile and water are used, the ratio (vol/vol) of acetonitrile to water is: about 3:1 to about 1:3; about 2:1 to about 1:2, about 1.5:1 to about 1:1.5, about 1.2:1 to about 1:1.2, or about 1:1.

Alternatively, Mesdopetam Besylate Form BS1 may be prepared by a process comprising stirring a suspension of Mesdopetam Besylate in isopropanol. Preferably, the suspension of Mesdopetam Besylate in isopropanol is prepared by combining Mesdopetam (preferably form B1), benzenesulfonic acid, and isopropanol. More particularly, the process comprises:
(i) providing a mixture of Mesdopetam, preferably form B1, and benzenesulfonic acid in isopropanol;
(ii) optionally stirring the mixture; and
(iii) optionally isolating crystalline form BS1 of Mesdopetam Besylate from the mixture.

In any aspect or embodiment of this process, in in step (i), the isopropanol may be present in an amount of: about 5 to about 25 ml per gram of Mesdopetam, about 8 to about 20 ml per gram of Mesdopetam, about 10 to about 15 ml per gram of Mesdopetam, or about 11 ml to about 17 ml per gram of Mesdopetam. In any embodiment of this process, the molar ratio of benzenesulfonic acid to Mesdopetam may be: about 1 to about 1.5 molar equivalents, about 1 to about 1.3 molar equivalents, about 1.0 to about 1.2 molar equivalents, about 1.0 to about 1.1 molar equivalents, or about 1.0 molar equivalents. In any embodiment of this process, the mixture in step (i) is preferably at room temperature. In any embodiment of this process, step (ii) comprises stirring the mixture for a sufficient time to prepare Mesdopetam besylate form BS1, preferably about 4 hours to about 72 hours, about 8 hours to about 48 hours, about 16 hours to about 36 hours, about 20 hours to about 30 hours, or about 24 hours. In any embodiment of this process, step (iii) comprises isolating the Mesdopetam besylate Form BS1 by any suitable method such as by decantation, centrifuge or filtration, preferably filtration. The Mesdopetam besylate Form BS1 may optionally be dried, for example under reduced pressure, and optionally elevated temperature, preferably at reduced pressure and at a temperature of: about 40° C. to about 90° C., about 55° C. to about 80° C., or about 60° C. to about 75° C.; optionally, the drying can be conducted for a sufficient time to obtain a dried solid, preferably about 1 hour to about 10 hours, about 2 hours to about 8 hours, about 3 hours to about 6 hours, or about 4 hours to about 6 hours.

The present disclosure encompasses Mesdopetam (L)-malate. The Mesdopetam (L)-malate is preferably in a solid form, and more preferably the Mesdopetam (L)-malate is crystalline. In any aspect or embodiment of the present disclosure, Mesdopetam (L)-malate may be isolated. Particularly, Mesdopetam (L)-malate according to any aspect or embodiment may be chemically pure. Crystalline Mesdopetam (L)-malate according to any aspect or embodiment may be polymorphically pure (i.e. containing a single crystalline form of Mesdopetam (L)-malate), preferably containing about 20% (w/w) or less, about 10% (w/w) or less, about 5% (w/w) or less, about 2% (w/w) or less, about 1% (w/w) or less, or about 0% of any other crystalline form of Mesdopetam (L)-malate, as measured, for example, by XRPD. In any aspect or embodiment of the present disclosure, Mesdopetam (L)-malate may be anhydrous.

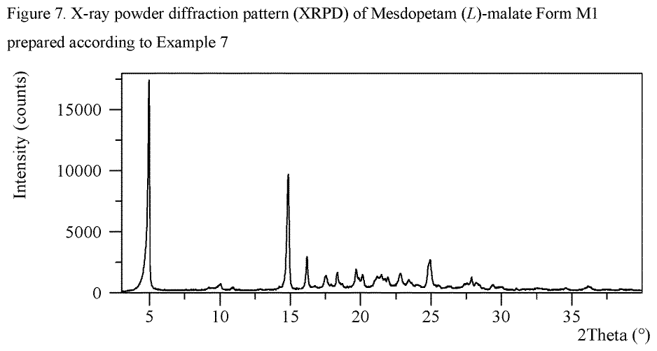

The present disclosure includes a crystalline polymorph Mesdopetam (L)-malate designated Form M1. The crystalline Form M1 of Mesdopetam (L)-malate may be characterized by data selected from one or more of the following: an X-ray powder diffraction pattern substantially as depicted in FIG. 7; an X-ray powder diffraction pattern having peaks at 5.0, 14.9, 16.2, 22.8 and 24.9 degrees 2-theta±0.2 degrees 2-theta; and combinations of these data.

Crystalline Form M1 of Mesdopetam (L)-malate may be further characterized by an X-ray powder diffraction pattern having peaks at 5.0, 14.9, 16.2, 22.8 and 24.9 degrees 2-theta±0.2 degrees 2-theta, and also having any one, two, three or four additional peaks selected from 17.5, 18.3, 19.7 and 23.4 degrees 2-theta±0.2 degrees 2-theta.

Crystalline Form M1 of Mesdopetam (L)-malate may be alternatively characterized by an X-ray powder diffraction pattern having peaks at 5.0, 14.9, 16.2, 17.5, 18.3, 19.7, 22.8, 23.4 and 24.9 degrees 2-theta±0.2 degrees 2-theta.

In any aspect or embodiment of the present disclosure, crystalline Form M1 of Mesdopetam (L)-malate may be isolated. Particularly, crystalline Form M1 of Mesdopetam (L)-malate according to any aspect or embodiment of the disclosure may be isolated.

In any aspect or embodiment crystalline form M1 of Mesdopetam (L)-malate may be chemically pure.

In any aspect or embodiment crystalline Form M1 of Mesdopetam (L)-malate may be polymorphically pure.

In any aspect or embodiment of the present disclosure, crystalline Form M1 of Mesdopetam (L)-malate may be anhydrous.

Crystalline Form M1 of Mesdopetam (L)-malate may be characterized by each of the above characteristics alone/or by all possible combinations, e.g., an XRPD pattern having peaks at 5.0, 14.9, 16.2, 22.8 and 24.9 degrees 2-theta±0.2 degrees 2-theta; an XRPD pattern as depicted in FIG. 7, and combinations thereof.

The present disclosure encompasses Mesdopetam hemi-(L)-malate. The Mesdopetam hemi-(L)-malate is preferably in a solid form, and more preferably the Mesdopetam-hemi (L)-malate is crystalline. In any aspect or embodiment of the present disclosure, Mesdopetam hemi-(L)-malate may be isolated. Particularly, Mesdopetam hemi-(L)-malate according to any aspect or embodiment may be chemically pure. Crystalline Mesdopetam hemi-(L)-malate according to any aspect or embodiment may be polymorphically pure (i.e. containing a single crystalline form of Mesdopetam hemi-(L)-malate), preferably containing about 20% (w/w) or less, about 10% (w/w) or less, about 5% (w/w) or less, about 2% (w/w) or less, about 1% (w/w) or less, or about 0% of any other crystalline form of Mesdopetam hemi-(L)-malate, as measured, for example, by XRPD. In any aspect or embodiment of the present disclosure, Mesdopetam-hemi (L)-malate may be anhydrous.

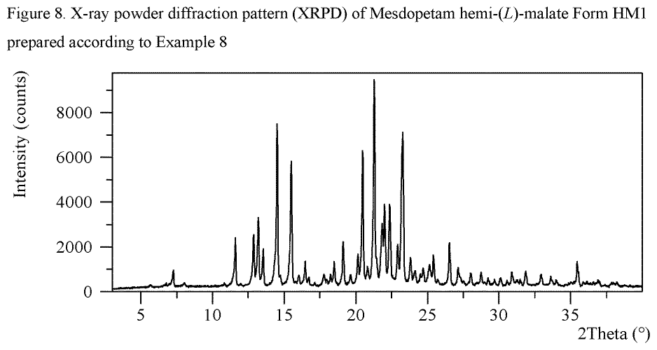

The present disclosure includes a crystalline polymorph Mesdopetam hemi-(L)-malate designated Form HM1. The crystalline Form HM1 of Mesdopetam hemi-(L)-malate may be characterized by data selected from one or more of the following: an X-ray powder diffraction pattern substantially as depicted in FIG. 8; an X-ray powder diffraction pattern having peaks at 14.5, 15.5, 20.5, 21.3 and 23.3 degrees 2-theta±0.2 degrees 2-theta; and combinations of these data.

Crystalline Form HMI of Mesdopetam hemi-(L)-malate may be further characterized by an X-ray powder diffraction pattern having peaks at 14.5, 15.5, 20.5, 21.3 and 23.3 degrees 2-theta±0.2 degrees 2-theta, and also having any one, two, three, four or five additional peaks selected from 11.6, 13.2, 19.1, 22.4 and 26.5 degrees 2-theta±0.2 degrees 2-theta.

Crystalline Form HMI of Mesdopetam hemi-(L)-malate may be alternatively characterized by an X-ray powder diffraction pattern having peaks at 11.6, 13.2, 14.5, 15.5, 19.1, 20.5, 21.3, 22.4, 23.3, and 26.5 degrees 2-theta±0.2 degrees 2-theta.

In any aspect or embodiment of the present disclosure, crystalline Form HM1 of Mesdopetam hemi-(L)-malate may be isolated. Particularly, crystalline Form HMI of Mesdopetam hemi-(L)-malate according to any aspect or embodiment of the disclosure may be isolated.

In any aspect or embodiment crystalline form HMI of Mesdopetam hemi-(L)-malate may be chemically pure.

In any aspect or embodiment crystalline Form HMI of Mesdopetam hemi-(L)-malate may be polymorphically pure.

In any aspect or embodiment of the present disclosure, crystalline Form HMI of Mesdopetam hemi-(L)-malate may be anhydrous.

Crystalline Form HMI of Mesdopetam hemi-(L)-malate may be characterized by each of the above characteristics alone/or by all possible combinations, e.g., an XRPD pattern having peaks at 14.5, 15.5, 20.5, 21.3 and 23.3 degrees 2-theta±0.2 degrees 2-theta; an XRPD pattern as depicted in FIG. 8, and combinations thereof.

Crystalline form HMI of Mesdopetam hemi-(L)-malate may be prepared by crystallisation from a mixture comprising Mesdopetam hemi-(L)-malate and a solvent comprising, acetonitrile, ethanol, methanol, acetone, or mixture thereof.

Mesdopetam hemi-(L)-malate may be prepared by reacting Mesdopetam with L-malic acid in any suitable solvent. The solvent is preferably selected at least enable dissolution of Mesdopetam and L-malic acid, and optionally enables precipitation of the resulting Mesdopetam hemi-(L)-malate (e.g. by cooling or evaporation). If the solvent does not readily enable precipitation of Mesdopetam hemi-(Z)-malate, an antisolvent for Mesdopetam hemi-(Z)-malate, for example a non-polar solvent such as an alkane, or a solvent selected from n-heptane, dichloromethane, acetone, or tetrahydrofuran, may be added to facilitate precipitation.

Crystalline form HMI of Mesdopetam hemi-(L)-malate may be prepared by crystallisation from a mixture comprising Mesdopetam hemi-(L)-malate and a solvent comprising acetonitrile, ethanol (96% ethanol/water or absolute ethanol), methanol, or mixture thereof. In any embodiment, the process may comprise:
(a) providing a solution of Mesdopetam hemi-(Z)-malate in a solvent comprising acetonitrile, ethanol, methanol or mixtures thereof;
(b) optionally cooling the mixture; and
(c) optionally isolating crystalline form HMI of Mesdopetam hemi-(L)-malate from the mixture.

In any embodiment of this process, the solution in step (a) may be prepared by combining Mesdopetam hemi-(L)-malate with the solvent. In any embodiment of this process, the solution in step (a), the solvent may be present in an amount of: about 5 to about 40 ml per gram of Mesdopetam hemi-(L)-malate, about 5 ml to about 35 ml per gram of Mesdopetam hemi-(L)-malate, about 7 to about 32 ml per gram of Mesdopetam hemi-(Z)-malate, or about 10 to about 30 ml per gram of Mesdopetam hemi-(L)-malate. In any embodiment of the process, the solution in step (a) may be at elevated temperature, preferably, at a temperature of: about 15° C. to about the reflux temperature, about 15° C. to about 100° C., about 18° C. to about 100° C., about 20° C. to about 98° C., or about 22° C. to about 98° C. In any embodiment, the solution in step (a) may be prepared by combining Mesdopetam hemi-(L)-malate with the heated solvent, or by combining Mesdopetam hemi-(L)-malate with the solvent at ambient temperature, and optionally heating the mixture to provide a solution. In any embodiment of the process, step (b) may comprise: cooling the mixture, preferably to about 5° C. to about 30° C., or about 10° C. to about 25° C. or about 20° C. to about 25° C. Preferably, in any embodiment of this process, the solution in step (a) is heated to a temperature of about 18° C. to about 60° C. (preferably about 22° C. to about 60° C.), and step (b) comprises cooling the mixture in step (a) to a temperature of: about 5° C. to about 35° C., about 10° C. to about 30° C., or room temperature. In any embodiment, the cooling may be active or passive, preferably passive (i.e. the mixture is allowed to cool under ambient conditions). In any embodiment of this process, the cooling is preferably conducted without stirring. In any embodiment of this process, step (c) may comprise isolating the solid by any suitable method such as by decantation, centrifuge or by filtration, preferably by filtration. The Mesdopetam hemi-(L)-malate form HMI may optionally be dried, for example under reduced pressure, and optionally elevated temperature, preferably at reduced pressure and at a temperature of: about 40° C. to about 90° C., about 55° C. to about 85° C., or about 60° C. to about 80° C.; optionally, the drying can be conducted for a sufficient time to obtain a dried solid, preferably about 1 hour to about 10 hours, about 2 hours to about 8 hours, about 3 hours to about 6 hours, or about 4 hours to about 6 hours.

According to any embodiment of the process, when the solvent is acetonitrile, the solution in step a) may be at a temperature of about 50° C. to about the reflux temperature, about 80° C. about 100° C., about 90° C. to about 100° C., or about the reflux temperature; and/or the acetonitrile is used in an amount of about 4 ml to about 20 ml, about 6 ml to 15 ml, about 8 ml to about 12 ml, or about 10 ml per gram of Mesdopetam Hemi-(L)-malate. According to any embodiment of the process, when the solvent is ethanol, step a) may be performed at a temperature of about 50° C. to about 80° C., about 60° C. to about 75° C., or about 65° C. to about 70° C.; and/or the ethanol is used in an amount of: about 15 ml to 35 ml, about 18 ml to about 32 ml, or about 20 ml to about 30 ml per gram of Mesdopetam Hemi-(L)-malate. According to any embodiment of the process, when the solvent is methanol, step a) may be performed at a temperature of about 18° C. to about 35° C., about 18° C. to about 30° C., about 20° C. to about 25° C. or at room temperature; and/or the methanol is used in an amount of about 5 ml to 20 ml, about 8 to about 15 ml, or about 10 ml per gram of Mesdopetam Hemi-(L)-malate.

Alternatively, Mesdopetam Hemi-(L)-malate Form HM1 may be prepared by a process comprising stirring a suspension of Mesdopetam Hemi-(L)-malate in acetone. Preferably, the suspension of Mesdopetam Hemi-(L)-malate in acetone is prepared by combining Mesdopetam (preferably form B1), L-malic acid, and acetone. More particularly, the process comprises:
(i) providing a mixture of Mesdopetam, preferably form B1, and (L)-malic acid in acetone;
(ii) optionally stirring the mixture; and
(iii) optionally isolating crystalline form HMI of Mesdopetam hemi-(L)-malate from the mixture.

In any aspect or embodiment of this process, in step (i), the acetone may be present in an amount of: about 5 to about 20 ml per gram of Mesdopetam, about 7 to about 18 ml per gram of Mesdopetam, about 10 to about 15 ml, or about 11 to about 13 ml per gram of Mesdopetam. In any embodiment of this process, the amount of (L)-malic acid to Mesdopetam may be: about 0.5 mole equivalents. In any embodiment of this process, the mixture in step (i) is preferably at room temperature. In any embodiment of this process, step (ii) comprises stirring the mixture for a sufficient time to prepare Mesdopetam hemi-(L)-malate form HM1, preferably about 4 hours to about 72 hours, about 8 hours to about 60 hours, about 18 hours to about 50 hours, about or about 24 hours to about 48 hours. In any embodiment of this process, step (iii) comprises isolating the Mesdopetam hemi-(L)-malate Form HM1 by any suitable method such as by decantation, centrifuge or filtration, preferably filtration. The Mesdopetam hemi-(L)-malate Form HM1 may optionally be dried, for example under reduced pressure, and optionally elevated temperature, preferably at reduced pressure and at a temperature of: about 40° C. to about 90° C., about 55° C. to about 80° C., or about 60° C. to about 75° C.; optionally, the drying can be conducted for a sufficient time to obtain a dried solid, preferably about 1 hour to about 10 hours, about 2 hours to about 8 hours, about 3 hours to about 6 hours, or about 4 hours to about 6 hours.

The present disclosure encompasses a process for preparing solid state forms of Mesdopetam, Mesdopetam salts and solid state forms thereof. The process includes preparing any one of the Mesdopetam salts, or solid state forms of Mesdopetam or salts thereof by the processes of the present disclosure, and converting that form to a different form of Mesdopetam or salts thereof. For example, the conversion can be done, for example, by a process comprising acidifying any one of the above described forms of Mesdopetam to prepare the corresponding salt. Alternatively, the solid state form of Mesdopetam of the present disclosure may be prepared from Mesdopetam salts. This conversion can be done, for example, by a process comprising basifying a Mesdopetam salt. In yet another example, the process may include preparing any one of the solid state forms of Mesdopetam salts of the present disclosure by the processes of the present disclosure, and converting it to said other form of Mesdopetam or Mesdopetam salt. The conversion can be done for example by a process comprising basifying any one of the above described forms of Mesdopetam salts and reacting the obtained Mesdopetam with an appropriate acid, to obtain the corresponding salt. Alternatively, the conversion can be done by salt switching, i.e., reacting any one of the forms of the Mesdopetam salts of the present disclosure with an acid having a pKa which is lower than that of the acid of the original salt.

The Mesdopetam salts and crystalline polymorphs of Mesdopetam or salts thereof as defined herein and the pharmaceutical compositions or formulations of the Mesdopetam salts, or crystalline polymorphs of Mesdopetam or salts thereof may be used as medicaments, such as for the treatment of patients with Parkinson's Disease Dyskinesia, levodopa-induced dyskinesias, or Parkinson's disease Psychosis.

The present disclosure also provides methods for the treatment of patients with levodopa-induced dyskinesias by administering a therapeutically effective amount of said Mesdopetam salts, or crystalline polymorphs of Mesdopetam or salts thereof of the present disclosure, or at least one of the above pharmaceutical compositions, to a subject suffering from Parkinson's Disease Dyskinesia, levodopa-induced dyskinesia, or Parkinson's disease Psychosis or otherwise in need of the treatment.

The present disclosure also provides uses of Mesdopetam salts, or crystalline polymorphs of Mesdopetam or salts thereof of the present disclosure, or at least one of the above pharmaceutical compositions, for the manufacture of medicaments for treating e.g., patients with Parkinson's Disease Dyskinesia, levodopa-induced dyskinesia, or Parkinson's disease Psychosis.

The present disclosure provides the above described Mesdopetam salts, or crystalline polymorphs of Mesdopetam or salts thereof for use in the preparation of pharmaceutical compositions comprising Mesdopetam or salts thereof and/or solid state forms thereof.

The present disclosure also encompasses the use of Mesdopetam salts, or crystalline polymorphs of Mesdopetam or salts thereof of the present disclosure for the preparation of pharmaceutical compositions of crystalline polymorph Mesdopetam or salts thereof and/or solid state forms thereof.

The present disclosure includes processes for preparing the above mentioned pharmaceutical compositions. The processes include combining any one or a combination of the Mesdopetam salts, or crystalline polymorphs of Mesdopetam or salts thereof of the present disclosure with at least one pharmaceutically acceptable excipient.

Pharmaceutical combinations or formulations of the present disclosure contain any one or a combination of the Mesdopetam salts, or solid state form of Mesdopetam or salts thereof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Mesdopetam or salts thereof and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Mesdopetam can be administered. Mesdopetam may be formulated for administration to a mammal, in embodiments to a human, by injection. Mesdopetam can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The Mesdopetam salts, and crystalline polymorph of Mesdopetam salts and the pharmaceutical compositions and/or formulations of Mesdopetam or Mesdopetam salts of the present disclosure can be used as medicaments, in embodiments for the treatment of patients with Diabetic Peripheral Neuropathic pain and/or Post-Herpetic Neuralgia.

The present disclosure also provides methods of treating of patients with Diabetic Peripheral Neuropathic pain and/or Post-Herpetic Neuralgia by administering a therapeutically effective amount of any one or a combination of the Mesdopetam salts, or crystalline polymorphs of Mesdopetam of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample is powdered in a mortar and pestle and applied directly on a silicon plate holder. The X-ray powder diffraction pattern was measured with Philips X'Pert PRO X-ray powder diffractometer, equipped with Cu irradiation source=1.54184 \acute{Å} (\hat{Å}ngström), X'Celerator (2.022° 2θ) detector. Scanning parameters: angle range: 3-40 deg., step size 0.0167, time per step 37 s, continuous scan.

The described peak positions were determined without using silicon powder as an internal standard in an admixture with the sample measured.

### EXAMPLES

### Preparation of Starting Materials

Mesdopetam hemitartrate can be prepared according to methods known from the literature, for example according to the disclosure in International Publication No. WO 2020/239568.

Mesdopetam hemitartrate may also be prepared according to the following procedure: 2.0 grams of Mesdopetam hemitartrate crude material was dissolved in 4 ml of solvent mixture ethanol/water (ratio 1:1) at 65° C. The solution was allowed to cool and crystallization occurred at 30° C. The suspension was allowed to cool to room temperature and vacuum filtered at room temperature. The solid was vacuum dried at 80° C. for 6 hours.

### Example 1: Preparation of Mesdopetam Crystal Form B1

1000 mg of Mesdopetam hemitartrate was dissolved in 5 ml of water at room temperature. Aqueous solution of sodium hydroxide (1 M) was added dropwise until pH of 11.7 was reached. The solution was extracted with 20 ml of dichloromethane and the organic layer was evaporated on rotavapour to obtain an oil. 3 ml of heptane was added to the oil and a suspension was observed after 20 minutes of mixing. The suspension was mixed for additional 5 hours and vacuum filtered. Obtained solid was analyzed and characterized by X-ray powder diffraction as Mesdopetam Form B1. The XRPD pattern is presented in FIG. 1.

### Example 2: Preparation of Mesdopetam Crystal Form B3

Mesdopetam form B1 (200 mg) was dissolved in cyclohexanone (1.5 mL) at room temperature. Solution was put into ice bath and crystallization occurred after 10 minutes of mixing. Suspension was additionally stirred for 3 hours and filtered. Obtained solid was dried in a vacuum oven at 80° C. for 12 hours. The obtained product was analyzed by XRPD and characterized as Mesdopetam form B3. The XRPD pattern is presented in FIG. 2.

### Example 3: Preparation of Mesdopetam Crystal Form B4

Mesdopetam form B1 (20 mg) was dissolved in cyclohexanone (0.2 mL) at room temperature. Prepared solution was left open at room conditions for solvent to evaporate. After crystallization occurred, crystals were filtrated off, analyzed by XRPD and characterized as Mesdopetam form B4. The XRPD pattern is presented in FIG. 3.

### Example 4: Preparation of Mesdopetam Mesylate Crystal Form MS1

70 mg of Mesdopetam form B1 and 20 μL of methanesulfonic acid were suspended in 1 ml of acetone at room temperature. Suspension was mixed for 4 days and vacuum filtered. Obtained solid was analyzed by XRPD and characterized as Mesdopetam mesylate form MS1. The XRPD pattern is presented in FIG. 4.

### Example 5: Preparation of Mesdopetam Mesylate Crystal Form MS2

### Procedure A

455 mg of Mesdopetam form B1 and 1.05 mL of methanesulfonic acid were suspended in 5 ml of 2-propanol at room temperature. Suspension was mixed for 2 days, vacuum filtered and vacuum dried at 60° C. for 4 hours. Obtained solid was analyzed by XRPD and characterized as Mesdopetam mesylate form MS2. The XRPD pattern is presented in FIG. 5.

### Procedure B

70 mg of Mesdopetam form B1 and 20 μL of methanesulfonic acid were suspended in 1 ml of water at room temperature. Solution was obtained after stirring for 5 minutes and it was left to evaporate. After 4 days solid was obtained. Obtained solid was analyzed by XRPD and identified as Mesdopetam mesylate form MS2.

### Procedure C

667 mg of Mesdopetam form B1 and 0.16 ml (1 eq) of methanesulfonic acid were suspended in 5 ml of 2-propanol at room temperature. Suspension was mixed for 1 day and vacuum filtered and dried at 80° C. for 6 hours in vacuum dryer. Obtained solid was analyzed by XRPD and identified as Mesdopetam mesylate form MS2. The material was vacuum dried at 80° C. for 6 hours, analyzed by XRPD and identified as Mesdopetam mesylate form MS2.

### Procedure D

50 mg of Mesdopetam mesylate was dissolved in 1 ml of Acetonitrile at 64° C. Solution was left to cool (without stirring) and crystallization occurred. The reaction mixture was vacuum filtered. Obtained solid was analyzed by XRPD and identified as Mesdopetam mesylate form MS2.

### Procedure E

50 mg of Mesdopetam mesylate was dissolved in 1 ml of Ethanol, 96% (or absolute) at 56° C. (70° C. with abs EtOH). Solution was left to cool (without stirring) and crystallization occurred. The reaction mixture was vacuum filtered. Obtained solid was analyzed by XRPD and identified as Mesdopetam mesylate form MS2.

### Procedure F

50 mg of Mesdopetam mesylate was dissolved in 0.5 ml of methanol at 45° C. Solution was left to cool (without stirring) and crystallization occurred. The reaction mixture was vacuum filtered. Obtained solid was analyzed by XRPD and identified as Mesdopetam mesylate form MS2.

### Procedure G

455 mg of Mesdopetam form B1 and 0.105 mL of methanesulfonic acid were suspended in 5 ml of 2-propanol at room temperature. Suspension was mixed for 2 days, vacuum filtered and vacuum dried at 60° C. for 4 hours. Obtained solid was analyzed by XRPD and characterized as Mesdopetam mesylate form MS2. The XRPD pattern is presented in FIG. 5.

### Example 6: Preparation of Mesdopetam Besylate Crystal Form BS1

### Procedure A

59 mg of Mesdopetam form B1 and 49 mg of benzenesulfonic acid were suspended in 1 ml of 2-propanol at room temperature. Suspension was mixed for 1 day, vacuum filtered and vacuum dried at 60° C. for 4 hours. Obtained solid was analyzed by XRPD and characterized as Mesdopetam besylate form BS1. The XRPD pattern is presented in FIG. 6.

### Procedure B

813 mg of Mesdopetam form B1 and 468 mg (1 eq) of benzenesulfonic acid were suspended in 9 ml of 2-propanol at room temperature. Suspension was mixed for 1 day and vacuum filtered. Obtained solid was analyzed by XRPD and identified as Mesdopetam besylate form BS1. The material was vacuum dried at 75° C. for 5 hours, analyzed by XRPD and identified as Mesdopetam besylate form BS1.

### Procedure C

50 mg of Mesdopetam besylate was dissolved in 1 ml of acetonitrile at 48° C. Solution was left to cool (without stirring) and crystallization occurred. The reaction mixture was vacuum filtered. Obtained solid was analyzed by XRPD and identified as Mesdopetam besylate form BS1.

### Procedure D

50 mg of Mesdopetam besylate was dissolved in 1 ml of ethanol, 96% at 57° C. (in ethanol, absolute at 59° C.). Solution was left to cool (without stirring) and crystallization occurred. The reaction mixture was vacuum filtered. Obtained solid was analyzed by XRPD and identified as Mesdopetam besylate form BS1.

### Procedure E

50 mg of Mesdopetam besylate was dissolved in 0.5 ml of methanol at room temperature. Solution was left to cool (without stirring) and crystallization occurred. The reaction mixture was vacuum filtered. Obtained solid was analyzed by XRPD and identified as Mesdopetam besylate form BS1.

### Procedure F

50 mg of Mesdopetam besylate was dissolved in 1.5 ml of 2-propanol at 51° C. Solution was left to cool (without stirring) and crystallization occurred. The reaction mixture was vacuum filtered. Obtained solid was analyzed by XRPD and identified as Mesdopetam besylate form BS1.

### Procedure G

50 mg of Mesdopetam besylate was dissolved in 0.25 ml of acetonitrile/water (1:1) at room temperature. Solution was left to cool (without stirring) and crystallization occurred. Obtained solid was analyzed by XRPD and identified as Mesdopetam besylate form BS1.

### Example 7: Preparation of Mesdopetam (L)-Malate Crystal Form M1

63 mg Mesdopetam form B1 and 37 mg of L-malic acid were suspended in 1 ml of acetone at room temperature. Suspension was dissolved after 5 minutes of mixing and it was put to ice bath for 1 day. Suspension was obtained after 1 day, vacuum filtered and vacuum dried at 60° C. for 4 hours. Obtained solid was analyzed by XRPD and characterized as Mesdopetam malate form M1. The XRPD pattern is presented in FIG. 7.

### Example 8: Preparation of Mesdopetam Hemi-(L)-Malate Crystal Form HM1

### Procedure A

80 mg of Mesdopetam form B1 and 20 mg of L-malic acid were suspended in 1 ml of acetone at room temperature. Suspension was mixed for 2 days, vacuum filtered and vacuum dried at 60° C. for 4 hours. Obtained solid was analyzed by XRPD and characterized as Mesdopetam hemi-L-malate form HM1. The XRPD pattern is presented in FIG. 8.

### Procedure B

483 mg of Mesdopetam base and 117 mg of L-malic acid (0.5 eq) were suspended in 6 ml of acetone at room temperature. Suspension was mixed for 1 day and vacuum filtered. Obtained solid was analyzed by XPRD and identified as Mesdopetam hemi-L-malate form HM1. Material was vacuum dried at 80° C. for 6 hours and analyzed by XRPD and identified as Mesdopetam hemi-L-malate form HM1.

### Procedure C

50 mg of Mesdopetam hemimalate was dissolved in 0.5 ml of acetonitrile at reflux temperature. Solution was left to cool (without stirring) and crystallization occurred. The reaction mixture was vacuum filtered. Obtained solid was analyzed by XRPD and identified as Mesdopetam hemi-L-malate form HM1.

### Procedure D

50 mg of Mesdopetam hemiamalate was dissolved in 1.5 ml of ethanol, absolute at 70° C. Solution was left to cool (without stirring) and crystallization occurred. The reaction mixture was vacuum filtered. Obtained solid was analyzed by XRPD and identified as Mesdopetam hemi-L-malate form HM1.

### Procedure E

50 mg of Mesdopetam hemimalate was dissolved in 1 ml of ethanol, 96% at 65° C. Solution was left to cool (without stirring) and crystallization occurred. The reaction mixture was vacuum filtered. Obtained solid was analyzed by XRPD and identified as Mesdopetam hemi-L-malate form HM1.

### Procedure F

50 mg of Mesdopetam hemimalate was dissolved in 0.5 ml of methanol at room temperature. Solution was left to cool (without stirring) and crystallization occurred. The reaction mixture was vacuum filtered. Obtained solid was analyzed by XRPD and identified as Mesdopetam hemi-L-malate form HM1.

### Example 9

### Stability of Mesdopetam Mesylate, Mesdopetam Besylate and Mesdopetam Hemi-(L)-Malate.

### Storage Stability at Different Relative Humidities at Ambient Temperature

Samples of Mesdopetam Mesylate Form MS2, Mesdopetam Besylate Form BS1 and Mesdopetam Hemi-(L)-Malate form HMI were subjected to conditions of different relative humidities at ambient temperature. XRPD analysis was performed on the samples after 7 days. The results are shown in Table 1 below.

TABLE 1

| | XRPD Analysis Results | | | |
|---|---|---|---|---|
| Starting | Relative humidity (25° C.) | | | |
| material | 20% | 40% | 60% | 80% |
| Mesdopetam | No | No | No | No |
| Mesylate | polymorphic | polymorphic | polymorphic | polymorphic |
| Form MS2 | conversion | conversion | conversion | conversion |
| Mesdopetam | No | No | No | No |
| Besylate | polymorphic | polymorphic | polymorphic | polymorphic |
| Form BS1 | conversion | conversion | conversion | conversion |
| Mesdopetam | No | No | No | No |
| Hemi-(L)- | polymorphic | polymorphic | polymorphic | polymorphic |
| Malate form | conversion | conversion | conversion | conversion |
| HM1 | | | | |

These results demonstrate that Mesdopetam Mesylate, Mesdopetam Besylate and Mesdopetam Hemi-(L)-Malate are especially stable to high and low relative humidity conditions and is particularly suitable for use in pharmaceutical dosage forms.

### Grinding Experiments

Samples of Mesdopetam Mesylate Form MS2, Mesdopetam Besylate Form BS1 and Mesdopetam Hemi-(L)-Malate form HM1 were subjected to strong grinding, and to solvent drop grinding in water, ethanol, ethyl acetate, isopropanol and acetone. In these experiments, about 20 mg of the sample is placed in a mortar and ground with a pestle for 5 minutes. The solvent, when used, as added to the crystalline material before grinding, in a volume of 10 microliters. XRPD analysis performed on each of the samples after the grinding experiment, confirmed no change in the starting material (Table 2):

TABLE 2

| | XRPD analysis results | | |
|---|---|---|---|
| | Mesdopetam Mesylate | Mesdopetam | Mesdopetam Hemi- |
| Condition | Form MS2 | Besylate Form BS1 | (L)-Malate Form HM1 |
| Strong grinding | No polymorphic | No polymorphic | No polymorphic |
| | conversion | conversion | conversion |
| Solvent-drop | No polymorphic | No polymorphic | No polymorphic |
| grinding (water) | conversion | conversion | conversion |
| Solvent-drop | No polymorphic | No polymorphic | No polymorphic |
| grinding (ethanol) | conversion | conversion | conversion |
| Solvent-drop | No polymorphic | No polymorphic | No polymorphic |
| grinding | conversion | conversion | conversion |
| (ethylacetate) | | | |
| Solvent-drop | No polymorphic | No polymorphic | No polymorphic |
| grinding | conversion | conversion | conversion |
| (isopropanol) | | | |
| Solvent-drop | No polymorphic | No polymorphic | No polymorphic |
| grinding (acetone) | conversion | conversion | conversion |

The results demonstrate that Mesdopetam Mesylate, Mesdopetam Besylate and Mesdopetam Hemi-(L)-Malate are resistant to polymorphic changes and are highly suitable for preparing pharmaceutical formulations.

### Thermal Stability

Samples of Mesdopetam Mesylate Form MS2, Mesdopetam Besylate Form BS1 and Mesdopetam Hemi-(L)-Malate form HMI were subjected to heating up to 100° C. for 30 minutes. XRPD analysis of the samples confirmed no change in the starting material (Table 3):

TABLE 3

| | Heating to 100° C., 30 minutes |
|---|---|
| Starting Material | XRPD analysis results |
| Mesdopetam Mesylate | No polymorphic conversion |
| Form MS2 | |
| Mesdopetam Besylate | No polymorphic conversion |
| Form BS1 | |
| Mesdopetam Hemi-(L)- | No polymorphic conversion |
| Malate Form HM1 | |

The above data shows that Mesdopetam Mesylate, Mesdopetam Besylate and Mesdopetam Hemi-(Z)-Malate are highly stable, showing no polymorphic conversion when heated to 100° C. for 30 minutes.

### Stability to Compression

Samples of Mesdopetam Mesylate Form MS2, Mesdopetam Besylate Form BS1 and Mesdopetam Hemi-(L)-Malate form HMI were subjected to a pressure of 1000 kg and 2000 kg (Atlas® Autopress hydraulic press, set to 1 or 2 tons). XRPD analysis was performed on the samples after 1-5 minutes. The results are shown in Table 4 below:

TABLE 4

| | XRPD analysis results | |
|---|---|---|
| Starting Material | Pressure (1000 kg) | Pressure (2000 kg) |
| Mesdopetam Mesylate | No polymorphic | No polymorphic |
| Form MS2 | conversion | conversion |
| Mesdopetam Besylate | No polymorphic | No polymorphic |
| Form BS1 | conversion | conversion |
| Mesdopetam Hemi-(L)- | No polymorphic | No polymorphic |
| Malate Form HM1 | conversion | conversion |

The results demonstrate the high stability of Mesdopetam Mesylate Mesdopetam Besylate and Mesdopetam Hemi-(L)-Malate to compression, and further confirm the suitability of these salts for preparing pharmaceutical dosage forms.

### Stability to Solvent Vapours

Samples of Mesdopetam Mesylate Form MS2, Mesdopetam Besylate Form BS1 and Mesdopetam Hemi-(L)-Malate form HMI were exposed to solvent vapours of acetone, ethanol, isopropanol, water and ethyl acetate at 25° C. XRPD analysis was performed on the samples after 7 days and after 30 days. The results are shown in Table 5 below:

TABLE 5

| Condition | XRPD analysis results | | |
|---|---|---|---|
| (7 days and | Mesdopetam Mesylate | Mesdopetam | Mesdopetam Hemi-(L)-Malate |
| 30 days) | Form MS2 | Besylate Form BS1 | Form HM1 |
| Acetone | No polymorphic | No polymorphic | No polymorphic |
| | conversion | conversion | conversion |
| Ethanol | No polymorphic | No polymorphic | No polymorphic |
| | conversion | conversion | conversion |
| Isopropanol | No polymorphic | No polymorphic | No polymorphic |
| | conversion | conversion | conversion |
| Water | No polymorphic | No polymorphic | No polymorphic |
| | conversion | conversion | conversion |
| Ethyl acetate | No polymorphic | No polymorphic | No polymorphic |
| | conversion | conversion | conversion |

Table 5

The above data further confirms the excellent stability of Mesdopetam Mesylate, Mesdopetam Besylate and Mesdopetam Hemi-(Z)-Malate.

1. A salt of Mesdopetam with an acid selected from methanesulfonic acid, benzenesulfonic acid, or (L)-malic acid.

2. The salt according to claim 1, which is selected from Mesdopetam mesylate, Mesdopetam besylate, or Mesdopetam hemi-(L)-malate, optionally wherein the salt is crystalline.

3. A crystalline form of Mesdopetam mesylate, designated form MS2, which is characterized by data selected from:
(a) an X-ray powder diffraction pattern having peaks at 7.8, 15.2, 19.6, 23.5 and 25.0 degrees 2-theta±0.2 degrees 2-theta;
(b) an X-ray powder diffraction pattern substantially as depicted in FIG. 5; and
(c) a combination of (a) and (b).

4. The crystalline form of Mesdopetam mesylate according to claim 3, which is characterized by an X-ray powder diffraction pattern having peaks at 7.8, 15.2, 19.6, 23.5 and 25.0 degrees 2-theta±0.2 degrees 2-theta, and also having any one, two, three, four or five additional peaks selected from 15.7, 17.2, 18.2, 20.2 and 20.9 degrees 2-theta±0.2 degrees 2-theta.

5. The crystalline form of Mesdopetam mesylate according to claim 3, which is characterized by an X-ray powder diffraction pattern having peaks at 7.8, 15.2, 15.7, 17.2, 18.2, 19.6, 20.2, 23.5 and 25.0 degrees 2-theta±0.2 degrees 2-theta.

6. The crystalline form of Mesdopetam mesylate according claim 3, which is characterized by an X-ray powder diffraction pattern having peaks at 7.8, 15.2, 15.7, 17.2, 18.2, 19.6, 20.2, 20.9, 23.5 and 25.0 degrees 2-theta±0.2 degrees 2-theta.

7. A crystalline form of Mesdopetam besylate, designated form BS1, which is characterized by data selected from:
(a) an X-ray powder diffraction pattern having peaks at 8.0, 15.0, 16.1, 18.2 and 22.6 degrees 2-theta±0.2 degrees 2-theta;
(b) an X-ray powder diffraction pattern substantially as depicted in FIG. 6; and
(c) a combination of (a) and (b).

8. The crystalline form of Mesdopetam besylate according to claim 7, which is characterized by an X-ray powder diffraction pattern having peaks at 8.0, 15.0, 16.1, 18.2 and 22.6 degrees 2-theta±0.2 degrees 2-theta, and also having any one, two, three or four additional peaks selected from 16.7, 17.7, 20.5 and 23.4 degrees 2-theta±0.2 degrees 2-theta.

9. The crystalline form of Mesdopetam besylate according to claim 7, which is characterized by an X-ray powder diffraction pattern having peaks at 8.0, 15.0, 16.1, 16.7, 17.7, 18.2, 20.5, 22.6 and 23.4 degrees 2-theta±0.2 degrees 2-theta.

10. A crystalline form of Mesdopetam hemi-(L)-malate, designated Form HM1, which is characterized by data selected from:
(a) an X-ray powder diffraction pattern having peaks at 14.5, 15.5, 20.5, 21.3 and 23.3 degrees 2-theta±0.2 degrees 2-theta;
(b) an X-ray powder diffraction pattern substantially as depicted in FIG. 8; and
(c) a combination of (a) and (b).

11. The crystalline form of Mesdopetam hemi-(L)-malate according claim 10, which is characterized by an X-ray powder diffraction pattern having peaks at 14.5, 15.5, 20.5, 21.3 and 23.3 degrees 2-theta±0.2 degrees 2-theta, and also having any one, two, three, four or five additional peaks selected from 11.6, 13.2, 19.1, 22.4 and 26.5 degrees 2-theta±0.2 degrees 2-theta.

12. The crystalline form of Mesdopetam hemi-(L)-malate according to claim 10, which is characterized by an X-ray powder diffraction pattern having peaks at 11.6, 13.2, 14.5, 15.5, 19.1, 20.5, 21.3, 22.4, 23.3, and 26.5 degrees 2-theta±0.2 degrees 2-theta.

13. The crystalline form of Mesdopetam mesylate according to claim 3, which is anhydrous.

14. (canceled)

15. The crystalline form of Mesdopetam mesylate according to claim 3, which is polymorphically pure; wherein the crystalline form of Mesdopetam mesylate contains: about 20% (w/w) or less, about 10% (w/w) or less, about 5% (w/w) or less, about 2% (w/w) or less, about 1% (w/w) or less, or about 0% of any other crystalline forms of the same compound, and/or wherein the crystalline form of Mesdopetam mesylate contains: about 20% (w/w) or less, about 10% (w/w) or less, about 5% (w/w) or less, about 2% (w/w) or less, about 1% (w/w) or less, or about 0% of any amorphous forms of the same compound.

16. (canceled)

17. (canceled)

18. A pharmaceutical composition or formulation comprising the crystalline form of Mesdopetam mesylate according to claim 3, and at least one pharmaceutically acceptable excipient, optionally in the form of a solid dosage form, including at least one of a capsule or tablet.

19. A process preparing a pharmaceutical composition or formulation according, the process comprising combining the crystalline form of Mesdopetam mesylate according to claim 3 with at least one pharmaceutically acceptable excipient.

20. (canceled)

21. (canceled)

22. A method of treating Parkinson's Disease Dyskinesia, levodopa-induced dyskinesia, or Parkinson's disease Psychosis, comprising administering a therapeutically effective amount of the crystalline form of Mesdopetam mesylate according to claim 3 to a subject in need of the treatment.

23. The crystalline form of Mesdopetam besylate according to claim 7, which is anhydrous.

24. The crystalline form of Mesdopetam besylate according to claim 7 which is polymorphically pure; wherein the crystalline form of Mesdopetam besylate contains: about 20% (w/w) or less, about 10% (w/w) or less, about 5% (w/w) or less, about 2% (w/w) or less, about 1% (w/w) or less, or about 0% of any other crystalline forms of the same compound, and/or wherein the crystalline form of Mesdopetam besylate contains: about 20% (w/w) or less, about 10% (w/w) or less, about 5% (w/w) or less, about 2% (w/w) or less, about 1% (w/w) or less, or about 0% of any amorphous forms of the same compound.

25. A pharmaceutical composition or formulation comprising the crystalline form of Mesdopetam besylate according to claim 7, and at least one pharmaceutically acceptable excipient, optionally in the form of a solid dosage form, including at least one of a capsule or tablet.

26. A process preparing a pharmaceutical composition or formulation, the process comprising combining the crystalline form of Mesdopetam besylate according to claim 7 with at least one pharmaceutically acceptable excipient.

27. A method of treating Parkinson's Disease Dyskinesia, levodopa-induced dyskinesia, or Parkinson's disease Psychosis, comprising administering a therapeutically effective amount of the crystalline form of Mesdopetam besylate according to claim 7 to a subject in need of the treatment.

28. The crystalline form of Mesdopetam hemi-(L)-malate according to claim 10, which is anhydrous.

29. The crystalline form of Mesdopetam hemi-(L)-malate according claim 10 which is polymorphically pure; wherein the crystalline form of Mesdopetam hemi-(L)-malate contains: about 20% (w/w) or less, about 10% (w/w) or less, about 5% (w/w) or less, about 2% (w/w) or less, about 1% (w/w) or less, or about 0% of any other crystalline forms of the same compound, and/or wherein the crystalline form of Mesdopetam hemi-(L)-malate contains: about 20% (w/w) or less, about 10% (w/w) or less, about 5% (w/w) or less, about 2% (w/w) or less, about 1% (w/w) or less, or about 0% of any amorphous forms of the same compound.

30. A pharmaceutical composition or formulation comprising the crystalline form of Mesdopetam hemi-(L)-malate according claim 10, and at least one pharmaceutically acceptable excipient, optionally in the form of a solid dosage form, including at least one of a capsule or tablet.

31. A process preparing a pharmaceutical composition or formulation, the process comprising combining the crystalline form of Mesdopetam hemi-(L)-malate according claim 10 with at least one pharmaceutically acceptable excipient.

32. A method of treating Parkinson's Disease Dyskinesia, levodopa-induced dyskinesia, or Parkinson's disease Psychosis, comprising administering a therapeutically effective amount of the crystalline form of Mesdopetam hemi-(L)-malate according claim 10 to a subject in need of the treatment.
